SEQUENCE LISTING (9 sequences)

SEQ ID NO: 1 - DNA, 178 bp, Homo sapiens
  variation (58): n=A,G, C or T
  variation (101): r=A or G, polymorphic site
gccggcgcttatttggcctggatggttcaggataatcaccttggaaaaaagatttgcnttcagtgcaaatccaaagtctcaaatgactttagcctctgcartaagttaatgttactttaaattgttacctgagcagtgaagccagctgcttccattggtgggtcatttttgctgtcac

SEQ ID NO: 2 - DNA, 179 bp, Homo sapiens
  variation (101): k=G or T, polymorphic site
agatatcaatggtgataccacgttcacgctcagctttcagtttatccaagacccaggcatacttgaaggagccctttcccatctgtaaggattaagagtckttacttggttactaaaacacaaactccagcttcaatttccttgtccccagcccttaattggcagtttccactttacaa

SEQ ID NO: 3 - DNA, 1837 bp, Homo sapiens
tttttcgcaacgggtttgccgccagaacacaggtgtcgtgaaaactacccctaaaagccaaaatgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaaggaagtcagcacttacattaagaaaattggctacaaccccgacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 18 bp, Artificial Sequence
  Forward primer for CCM108
atgactttagcctctgca

SEQ ID NO: 5 - DNA, 19 bp, Artificial Sequence
  Reverse primer for CCM108
atgactttagcctctgcaa

SEQ ID NO: 6 - DNA, 20 bp, Artificial Sequence
  Extension primer for CCM108
atgactttagcctctgcagt

SEQ ID NO: 7 - DNA, 21 bp, Artificial Sequence
  Forward primer for CCM128
tgtgttttagtaaccaagtaa

SEQ ID NO: 8 - DNA, 22 bp, Artificial Sequence
  Reverse primer for CCM128
tgtgttttagtaaccaagtaac

SEQ ID NO: 9 - DNA, 23 bp, Artificial Sequence
  Extension primer for CCM128
tgtgttttagtaaccaagtaaag

(19) United States
(12) Patent Application Publication
(10) Pub. No.: US 2006/0281123 A1
(43) Pub. Date: Dec. 14, 2006

(54) Polynucleotide comprising single-nucleotide polymorphism associated with colorectal cancer, microarray and diagnostic kit comprising the same, and method of diagnosing colorectal cancer using the same

(76) Inventors: Min-sun Kim, Hwaseong-si (KR); Sung-woo Hong, Yongin-si (KR); Jae-Heup Kim, Seoul (KR); Yeon-Su Lee, Goyang-si (KR); Ki-eun Klm, Seoul (KR); Tae-Jin Ahn, Seoul (KR)

Correspondence Address: CANTOR COLBURN, LLP, 55 GRIFFIN ROAD SOUTH, BLOOMFIELD, CT, 06002, US

(21) Appl. No.: 11/451,665

(22) Filed: Jun. 13, 2006

(30) Foreign Application Priority Data
Jun. 14, 2005 (KR) 10-2005-0051119

(51) Int. Cl. C12Q 1/68; C07H 21/04; C12M 1/34

(52) U.S. Cl. 435/6; 536/24.3; 435/287.2

(57) ABSTRACT

Provided are a polynucleotide for diagnosis or treatment of colorectal cancer including at least 10 contiguous nucleotides of a nucleotide sequence selected from the group consisting of nucleotide sequences of SEQ ID NOS: 1 and 2 and including a nucleotide at position 101 of the nucleotide sequence, or the complement thereof; a microarray having a substrate on which the polynucleotide is immobilized; a diagnostic kit for the detection of colorectal cancer including the polynucleotide; and a method of detecting colorectal cancer using the polynucleotide.

### CROSS-REFERENCE TO RELATED PATENT APPLICATION

This application claims the benefit of Korean Patent Application No. 10-2005-0051119, filed on Jun. 14, 2005, in the Korean Intellectual Property Office, the disclosure of which is incorporated herein in its entirety by reference.

### BACKGROUND OF THE INVENTION

1. Field of the Invention

The present invention relates to a polynucleotide associated with colorectal cancer, a microarray and a diagnostic kit including the same, and a method of diagnosing colorectal cancer.

2. Description of the Related Art

1. Incidence of colorectal cancer

Incidence of colorectal cancer has increased in American and European persons who frequently consume meat or other foods containing animal fat. In particular, in America, colorectal cancer is the second most common cancer in both incidence and death rate. Colorectal cancer incidence in Asian countries including Korea and Japan is lower than that in Western countries but has recently increased due to rapid Westernization of diet. According to a recent report (1997), in Korea, colorectal cancer is the fourth most common cancer.

Like other cancers affecting other organs, colorectal cancer frequently occurs in adults over 50 years of age but can also affect younger people.

2. Causative and risk factors of colorectal cancer

The exact cause of colorectal cancer is not known. However, it is well known that familial adenomatous polyposis, idiopathic nonspecific ulcerative colitis, colonic polyp, and rectal polyp, in particular, villous adenoma can turn to cancer. Although there is no conclusive evidence of a hereditary link to colorectal cancer, it is suspected that about 10-30% of colorectal cancer cases are dominated by a hereditary factor.

The incidence of colorectal cancer is more frequent in Western people than in Eastern people. Such an increased incidence of colorectal cancer is suspected to be associated with higher consumption of animal fat and meat in Western diets. That is, consumption of animal fat and meat produces less stool and the stool also stays in the large intestine for a longer time, relative to consumption of fiber-rich foods such as vegetables or grains. Higher consumption of animal fat affects bacteria that normally live in the healthy large intestine. Furthermore, if the stool stays in the large intestine for a long time, carcinogens are easily generated in the large intestine and thus greater exposure of colorectal cells to the carcinogens is caused. This explains the increased incidence of colorectal cancer. Epidemiological studies reveal that there is a relationship between the consumption of animal fat and meat and the incidence of colorectal cancer.

3. Symptoms of colorectal cancer

Colorectal cancer has no specific symptoms. However, colorectal cancer involves various symptoms according to the affected region or the level of advancement, in addition to common cancer symptoms such as weight loss. For example, when cancer is caused in the descending colon adjacent to the anus, the sigmoid colon, or the rectum, common symptoms include the following: blood in the stool, a change in bowel habits (repetition of diarrhea and constipation), stool narrower than usual, feeling that the bowel does not empty completely, or stomachache. When cancer is caused in the ascending colon, anemia (dizziness, vomiting, anorexia, fatigue, difficulty in breathing, etc.) due to unperceivable, chronic blood loss in the stool is caused.

In addition, as colorectal cancer develops, a gradual narrowing of the large intestine's inner passageway causes intestinal obstruction. Occasionally, abdominal tumor mass may be found, or the spread to distant organs, such as the liver or lungs, may occur.

4. Diagnosis of colorectal cancer

(1) Fecal occult blood test: the fecal occult blood test is a simple screening test to detect colorectal cancer. However, since this test can have a false-positive result due to other factors, it is not an absolute test for colorectal cancer.

(2) Tumor marker assay: the tumor marker assay is a blood test that looks for a carcinoembryonic antigen (CEA). About 50% of colorectal cancer patients undergo an increase in the CEA level. However, the increase in the CEA level does not necessarily prove the existence of colorectal cancer. Nevertheless, since a high CEA level indicates a high likelihood of colorectal cancer, a precise examination is additionally required for persons with a high CEA level. CEA is also helpful in evaluating the recurrence of colorectal cancer after treatment.

(3) Barium enema examination: the barium enema examination is radiation screening and detection of colorectal cancer based on a change in the outline of the mucosal membrane of the large intestine. Since this test shows the entire outline of the large intestine, it is helpful in detecting the location of cancer before surgery.

(4) Endoscopic examination: the endoscopic examination is divided into two groups: a short endoscopic examination to view the sigmoid colon and a long endoscopic examination to view the entire large bowel including the appendix. The endoscopic examination has a higher diagnostic accuracy than the barium enema examination. The endoscopic examination is an essential test for diagnosis of colorectal cancer since it enables histological examination, and thus a final diagnosis can be made by the histological examination, and polyps can be removed.

(5) Ultrasonic and computed tomography (CT) scan of the abdomen: when colorectal cancer is diagnosed by barium enema examination or endoscopic examination, an ultrasonic and CT scan show the localized stage and distant metastasis of the colorectal cancer.

(6) CEA and serologic tumor marker assay

For early diagnosis of colorectal cancer, various proteins, including glycoproteins, have been widely studied as promising tumor marker candidates. However, colorectal cancer-specific tumor markers have not been found to date. Currently, CEA is widely used in determining an advanced stage of colorectal cancer before surgery and evaluating the recurrence of colorectal cancer after surgery. However, CEA is not suitable for cancer patients with no symptoms.

5. Stage and treatment of colorectal cancer

According to the Dukes' classification, the stage of colorectal cancer is classified as A, B, C, or D according to the degree of invasion into the mucosal membrane of the large intestine, the degree of lymph node metastasis, and whether it has spread to other distant organs. Like other cancers, the stage of colorectal cancer is determined after surgery, and the treatment and prognosis of colorectal cancer vary according to the stage of colorectal cancer.

(1) Endoscopic treatment

Currently, endoscopic examination is regarded as an essential test for diagnosis of colorectal cancer, and at the same time, plays an important role in prevention or treatment of colorectal cancer. During endoscopic examination, polyps that may develop into cancer can be removed, thereby reducing the incidence of colorectal cancer. At the same time, colorectal cancer patients with small tumor mass like polyps can be simply treated by endoscopic resection.

(2) Surgical treatment

Surgery is a primary treatment for colorectal cancer and has a significant effect on the treatment result. The surgical treatment depends on the region affected by cancer. For colon cancer, the affected sections of the colon and surrounding lymph nodes are removed, and the remaining sections of the colon are then re-connected. For rectal cancer, if rectal cancer is located far away from the anus, only the cancer is removed with no removal of the anus. On the other hand, if rectal cancer is located close to the anus, the anus is removed with the cancer and an artificial anus is reconstructed.

(3) Radiotherapy

For rectal cancer, radiotherapy, together with drug therapy, may be performed after surgery according to the stage of the cancer. The radiotherapy may be given five days a week for 5-6 weeks and can reduce the risk of local recurrence and lymph node metastasis in the pelvis.

(4) Drug therapy

After surgery, when colorectal cancer is diagnosed to be in stage B, drug therapy is used in some cases. However, since drug therapy for stage B colorectal cancer is not a standard treatment, surgery may be followed by only periodic observation and examination. However, for stage C colorectal cancer, drug therapy for six months to one year is used as standard treatment. For colorectal cancer at stage D (terminal stage), drug therapy is used in spite of remarkably insignificant therapeutic effects since other therapies have failed.

6. Treatment result

The 5-year survival rate for colorectal cancer after surgery is as follows: 90% for stage A, 80% for stage B, 45% for stage C, and less than 10% for stage D. Like other cancers, the 5-year survival rate for colorectal cancer is greatly reduced as colorectal cancer advances. Therefore, early diagnosis and treatment of colorectal cancer are very important.

7. Prevention

Exact causes of colorectal cancer (colon cancer and rectal cancer) have not been found. It is known that high consumption of animal fat or meat is probably associated with an increased risk of colorectal cancer. Thus, a reduced intake of animal fat and a balanced diet of fresh vegetables and fiber-rich foods are recommended. Furthermore, it is advisable to avoid high consumption of foods containing chemicals such as dark pigments and preservatives.

When diseases closely associated with colorectal cancer, i.e., familial adenomatous polyposis, idiopathic nonspecific ulcerative colitis, colonic polyp, and rectal polyp are found, much interest and periodic examination are required to prevent colorectal cancer.

As described above, CEA is generally known as a colorectal cancer-specific marker. However, CEA has many limitations in early diagnosis of colorectal cancer.

eEF1A1 encodes an isoform of the alpha subunit of the elongation factor-1A complex. eEF1A1 is a pentamer which mediates the binding of cognate aminoacyl-tRNA to the A-site of the ribosome and its subsequent release. eEF1A1 is activated upon GTP binding. The human eEF1A1 gene includes, for example, a gene with a reference cDNA sequence corresponding to SEQ ID NO:3 or a reference genomic sequence corresponding to GenBank No. NC_{—}000006.10.

It was known that human eEF1A1 gene is upregulated or overexpressed in various cancer tissues, for example, in tumor tissues of the stomach, liver, pancreas, breast, lung, prostate, and colon (Science, 1997, May 23; 276 (5316): 1268-72). However, it has not been known whether single-nucleotide polymorphisms (SNPs) in the human eEF1A1 gene are associated with colorectal cancer.

SNPs take the form of single-nucleotide variations between individuals of the same species. When SNPs occur in protein coding sequences, any one of the polymorphic forms may give rise to the expression of a defective or a variant protein. On the other hand, when SNPs occur in non-coding sequences, some of these polymorphisms may result in the expression of defective or variant proteins (e.g., as a result of defective splicing). Other SNPs have no phenotypic effects.

It is known that human SNPs appear at a frequency of 1 in about 1,000 bp. When such SNPs induce a phenotypic expression such as a disease, polynucleotides containing the SNPs can be used as primers or probes for diagnosis of the disease. Currently, research into the nucleotide sequences and functions of SNPs is being conducted by many research institutes. The nucleotide sequences and other experimental results of the identified human SNPs have been collated into a database to be easily accessible. Even though findings available to date show that specific SNPs exist on human genomes or cDNAs, phenotypic effects of such SNPs have not been revealed. Functions of most SNPs have not yet been discovered.

Therefore, the present inventors found that specific SNPs in the human eEF1A1 gene were associated with colorectal cancer.

### SUMMARY OF THE INVENTION

The present invention provides a polynucleotide containing single-nucleotide polymorphism (SNP) associated with colorectal cancer.

The present invention also provides a microarray and a diagnostic kit for the detection of colorectal cancer, each of which includes the polynucleotide containing SNP associated with colorectal cancer.

The present invention also provides a method of diagnosing colorectal cancer in an individual. The method comprises determining a nucleotide at polymorphic site CCM108 or CCM128 in the eEF1A1 gene. The method can use the polynucleotides associated with colorectal cancer.

### DETAILED DESCRIPTION OF THE INVENTION

The present invention provides a polynucleotide for diagnosis or treatment of colorectal cancer including at least 10 contiguous nucleotides of a nucleotide sequence selected from the group consisting of nucleotide sequences of SEQ ID NOS: 1 and 2, derived from human eEF1A1 gene, and including a nucleotide at the polymorphic site (position 101) of the nucleotide sequence, or a complementary polynucleotide thereof.

The polynucleotide includes at least 10 contiguous nucleotides containing the polymorphic site of SEQ ID NO: 1 or SEQ ID NO: 2. The polynucleotide may be 10 to 400 nucleotides in length, specifically 10 to 100 nucleotides in length, and more specifically 10 to 50 nucleotides in length. The polymorphic site of each nucleotide sequence of SEQ ID NOS: 1 and 2 is at position 101.

Each nucleotide sequence of SEQ ID NOS: 1 and 2 is a polymorphic sequence. A polymorphic sequence refers to a nucleotide sequence containing a polymorphic site at which single-nucleotide polymorphism (SNP) occurs. A polymorphic site refers to a position of the polymorphic sequence at which SNP occurs. Each nucleotide sequence of SEQ ID NOS: 1 and 2 may be DNA or RNA. All or only part of the polymorphic sequence flanking the polymorphic site can be used by a practitioner in the art to identify the SNP in a nucleic acid. Identification can be, for example, by sequence alignment or by an experimental method such as hybridization. The parameters, such as the length of flanking sequence, needed to identify the SNP in a nucleic acid sequence are known.

In the present invention, each polymorphic site (position 101) of the polymorphic sequences of SEQ ID NOS: 1 and 2 is associated with colorectal cancer. This was confirmed by DNA nucleotide sequence analysis of blood samples from colorectal cancer patients and normal persons. The sequence analysis results of genotype occurrences in colorectal cancer patients (case) and normal persons are shown in Table 1.

TABLE 1

| | | | Case | | | Normal | | |
|---|---|---|---|---|---|---|---|---|
| SNP site | rs # | Gene | AA | Aa | aa | AA | Aa | aa |
| CCM108 | 1874230 | eEF1A1 | 84 | 116 | 15 | 140 | 118 | 30 |
| CCM128 | 2073466 | eEF1A1 | 84 | 113 | 32 | 139 | 116 | 37 |

In Table 1, nucleotides found at polymorphic site CCM108 are A or G; A is the dominant nucleotide (denoted as “A” in Table 1) and G is the recessive nucleotide (denoted “a” in Table 1). Nucleotides found at polymorphic site CCM128 are G or T, with G being the dominant nucleotide (“A” in Table 1) and T being the recessive nucleotide (“a” in Table 1).

The data shown in Table 1 was statistically analyzed for association of the genotypes of polymorphic sequences, SEQ ID NOS: 1 and 2, with colorectal cancer. The statistical results and the characteristics of the polymorphic sequences are summarized in Table 2.

TABLE 2

| | Role of | | | X² | recessive | recessive | recessive | recessive | recessive |
|---|---|---|---|---|---|---|---|---|---|
| Gene | SNP | rs# | SNP site | (95%) | OR | OR LB | OR UB | association | power |
| eEF1A1 | Intron | 1874230 | CCM108 | 0.033 | 0.630 | 0.441 | 0.899 | Positive | 0.312 |
| | (boundary) | (SEQ ID | | | | | | | |
| | | NO: 1) | | | | | | | |
| eEF1A1 | Intron | 2073466 | CCM128 | 0.038 | 0.638 | 0.448 | 0.908 | Positive | 0.301 |
| | (boundary) | (SEQ ID | | | | | | | |
| | | NO: 2) | | | | | | | |

In Table 1 or 2, rs # represents the SNP identification number assigned by NCBl dbSNP database.

Power represents the degree of data confidence.

Odds ratio (OR) represents the ratio of the probability of recessive genotypes in the case group to the probability of recessive genotypes in the normal group. Herein, “recessive genotypes” for the SNP site CCM108 (corresponding, for example, to the polymorphism at position 101 of SEQ ID NO: 1) are AG and GG and “recessive genotypes” for the SNP site CCM128 (corresponding, for example, to the polymorphism at position 101 of SEQ ID NO: 2) are GT and TT. In the present invention, the Mantel-Haenszel odds ratio method was used. Upper bound (UB) and lower bound (LB) of OR represent the 95% confidence interval limits for the odds ratio. When 1 falls within the 95% confidence interval limits, it is considered that there is insignificant association of recessive genotypes with the disease.

As shown in Tables 1 and 2, using the chi-square test of the polymorphic markers of CCM108 and CCM128 of the present invention, the chi-squared values are 0.033 and 0.038 for a 95% confidence, respectively. This shows that there are significant differences between expected values and measured values in allele occurrence frequencies in the polymorphic markers CCM108 and CCM128. The odds ratio for the SNP site CCM108 is 0.630 (with the 95% confidence limits ranging from 0.441 to 0.899) and for SNP site CCM 128, the odds ratio is 0.638 (95% confidence limits ranging from 0.448 to 0.908). These odds ratios show that the SNP sites CCM108 and CCM128, represented by the polymorphic markers of SEQ ID NOS: 1 and 2, are associated with colorectal cancer. When a recessive genotype exists at SNP site CCM108 or CCM128 in the eEF1A1 gene, a likelihood of colorectal cancer is high.

The present invention also provides an allele-specific polynucleotide for diagnosis of colorectal cancer, which is hybridized with a polynucleotide including at least 10 contiguous nucleotides containing a polymorphic site of a nucleotide sequence selected from the group consisting of nucleotide sequences of SEQ ID NOS: 1 and 2, or the complement thereof.

The allele-specific polynucleotide refers to a polynucleotide specifically hybridized with each allele. That is, an allele-specific polynucleotide has the ability to distinguish the alternative nucleotides that can occur at the polymorphic sites within the polymorphic sequences of SEQ ID NOS: 1 and 2 and specifically hybridize with one of the nucleotides. The hybridization is performed under stringent conditions, for example, a salt concentration of 1 M or less and a temperature of 25° C. or more. For example, conditions of 5×SSPE (750 mM NaCl, 50 mM Na Phosphate, 5 mM EDTA, pH 7.4) and 25-30° C. are suitable for allele-specific probe hybridization.

In the present invention, the allele-specific polynucleotide may be a primer. As used herein, the term “primer” refers to a single-stranded oligonucleotide that acts as a starting point of template-directed DNA synthesis under appropriate conditions, for example in a buffer containing four different nucleoside triphosphates and polymerase such as DNA or RNA polymerase or reverse transcriptase and an appropriate temperature. The appropriate length of the primer may vary according to the purpose of use, generally 15 to 30 nucleotides. Generally, a shorter primer molecule requires a lower temperature to form a stable hybrid with a template. A primer sequence is not necessarily completely complementary with a template but must be complementary enough to hybridize with the template. Preferably, the 3′ end of the primer is aligned with a nucleotide (position 101) of each polymorphic site of SEQ ID NOS: 1 and 2. The primer is hybridized with a target DNA containing a polymorphic site and starts an allelic amplification in which the primer exhibits complete homology with the target DNA. The primer is used in pair with a second primer hybridizing with an opposite strand. Amplified products are obtained by amplification using the two primers, which means that there is a specific allelic form. The primer of the present invention includes a polynucleotide fragment used in a ligase chain reaction (LCR).

In the present invention, the allele-specific polynucleotide may be a probe. As used herein, the term “probe” refers to a hybridization probe, that is, an oligonucleotide capable of binding sequence-specifically with a complementary strand of a nucleic acid. Such a probe may be a peptide nucleic acid as disclosed in Science 254, 1497-1500 (1991) by Nielsen et al. The probe according to the present invention is an allele-specific probe. In this regard, when there are polymorphic sites in nucleic acid fragments derived from two members of the same species, the probe is hybridized with DNA fragments derived from one member but is not hybridized with DNA fragments derived from the other member. In this case, hybridization conditions should be stringent enough to allow hybridization with only one allele by significant difference in hybridization strength between alleles. Preferably, the central portion of the probe, that is, position 7 for a 15 nucleotide probe, or position 8 or 9 for a 16 nucleotide probe, is aligned with each polymorphic site of the nucleotide sequences of SEQ ID NOS: 1 and 2. Therefore, a significant difference in hybridization between alleles may be caused. The probe of the present invention can be used in diagnostic methods for detecting alleles. The diagnostic methods include nucleic acid hybridization-based detection methods, e.g., southern blot. In a case where DNA chips are used for the nucleic acid hybridization-based detection methods, the probe may be provided as an immobilized form on a substrate of a DNA chip.

The present invention also provides a microarray for the detection of colorectal cancer, including the polynucleotide according to the present invention or the complementary polynucleotide thereof. The polynucleotide of the microarray may be DNA or RNA. The microarray is the same as a common microarray except that it includes the polynucleotide of the present invention.

The present invention also provides a diagnostic kit for the detection of colorectal cancer including the polynucleotide of the present invention. The diagnostic kit may include reagents necessary for polymerization, e.g., dNTPs, various polymerases, and a colorant, in addition to the polynucleotide according to the present invention.

The present invention also provides a method of diagnosing colorectal cancer in an individual, which includes determining a nucleotide present in the individual at a polymorphic site, wherein the polymorphic site corresponds to position 101 within a polynucleotide of SEQ ID NO: 1 or 2, or the complement thereof. Here, when the determined nucleotide at the polymorphic site is the same as the recessive nucleotide for the polymorphic site, presented in Table 2, it is determined that the individual has a higher likelihood of being diagnosed as at risk of developing colorectal cancer.

A method of diagnosing colorectal cancer in an individual can also comprise determining the presence or absence in the individual of a recessive nucleotide at polymorphic site CCM108 or polymorphic site CCM128 in the eEF1A1 gene. The presence of a recessive nucleotide at CCM108 or CCM128 identifies the individual as having a higher likelihood of being at risk of developing colorectal cancer.

The method of diagnosing colorectal cancer in an individual can further comprise determining whether the individual is homozygous or heterozygous for the recessive nucleotide at polymorphic site CCM108 or polymorphic site CCM128 in the eEF1A1 gene.

The method of diagnosing colorectal cancer in an individual can also include obtaining a nucleic acid sample from the individual. A nucleic acid sample can be obtained from any appropriate biological sample from the individual. For example, the nucleic acid sample can be obtained from a blood sample or a buccal swab.

The operation of isolating the nucleic acid sample from the individual may be performed using any known DNA isolation method. For example, the nucleic acid sample can be isolated by amplifying a target nucleic acid by polymerase chain reaction (PCR) followed by purification. In addition to PCR, there may be used LCR (Wu and Wallace, Genomics 4, 560 (1989), Landegren et al., Science 241, 1077 (1988)), transcription amplification (Kwoh et al., Proc. Natl. Acad. Sci. USA 86, 1173 (1989)), self-sustained sequence replication (Guatelli et al., Proc. Natl. Acad. Sci. USA 87, 1874 (1990)), or nucleic acid sequence based amplification (NASBA). The last two methods are related with isothermal reaction based on isothermal transcription and produce 30 or 100-fold RNA single strands and DNA double strands as amplification products.

According to an embodiment of the present invention, the operation of determining the nucleotide at a polymorphic site includes contacting the nucleic acid sample with a microarray on which polynucleotides for diagnosis or treatment of colorectal cancer are immobilized such that specific hybridization occurs; and detecting the hybridization result. The polynucleotides for diagnosis or treatment of colorectal cancer can comprise at least 10 contiguous nucleotides of SEQ ID NO: 1 or 2, wherein the at least 10 contiguous nucleotides comprise a nucleotide at the polymorphic site (position 101); or the complementary polynucleotides thereof.

A microarray and a method of manufacturing a microarray by immobilizing a probe polynucleotide on a substrate are well known in the pertinent art. Immobilization of a probe polynucleotide associated with colorectal cancer of the present invention on a substrate can be easily performed using a conventional technique. Hybridization of nucleic acids on a microarray and detection of the hybridization result are also well known in the pertinent art. For example, the detection of the hybridization result can be performed by labeling a nucleic acid sample with a labeling material generating a detectable signal, such as a fluorescent material (e.g., Cy3 and Cy5), hybridizing the labeled nucleic acid sample onto a microarray, and detecting a signal generated from the labeling material.

According to another embodiment of the present invention, as a result of the determination of a nucleotide sequence of polymorphic site CCM108 or CCM128 in the eEF1A1 gene, when genotypes at CCM108 (corresponding, for example, to the polymorphism at position 101 of SEQ ID NO: 1) are recessive genotypes AG and GG and/or genotypes at CCM128 (corresponding, for example, to the polymorphism at position 101 of SEQ ID NO: 2) are recessive genotypes GT and TT, it is determined that the individual has a higher likelihood of being diagnosed as a colorectal cancer patient or as at risk of developing colorectal cancer.

Hereinafter, the present invention will be described more specifically by Examples. However, the following Examples are provided for illustrative purposes only and are not intended to limit the present invention.

### EXAMPLES

### Example 1

### Analysis of Occurrence Frequency of SNPs of eEF1A1 Gene

In this Example, DNA samples were extracted from blood streams of a patient group consisting of Korean persons that had been diagnosed as colorectal cancer patients and had been undergoing treatment and a normal group consisting of Korean persons which were of the same age as those in the patient group and had no colorectal cancer symptoms. Allele occurrence frequencies of SNPs in the eEF1A1 gene were evaluated. SNPs used in Example 1 were rs1874230 and rs2073466 selected from a known database (NCBl dbSNP:http://www.ncbi.nim.nih.gov/SNP/). Primers hybridizing with sequences around the selected SNPs were used to assay nucleotides of SNPs in the DNA samples.

TABLE 3
The number of samples used in analysis

| SNP site | Case | Normal |
|---|---|---|
| CCM108 | 225 | 288 |
| CCM128 | 229 | 292 |

TABLE 4
Allele in SNP site

| SNP site | Alleles | Dominant allele (A) | Recessive allele (a) |
|---|---|---|---|
| CCM108 | A/G | A | G |
| CCM128 | A/C | G | T |

1. Preparation of DNA samples

DNA samples were extracted from blood streams of colorectal cancer patients and normal persons. DNA extraction was performed according to a known extraction method (Molecular cloning: A Laboratory Manual, p 392, Sambrook, Fritsch and Maniatis, 2nd edition, Cold Spring Harbor Press, 1989) and the specification of a commercial kit manufactured by Centra system. Among extracted DNA samples, only DNA samples having a purity (measured by A₂₆₀/A₂₈₀ nm ratio) of at least 1.7 were used.

2. Amplification of target DNAs

Target DNAs, which were predetermined DNA regions containing SNPs to be analyzed, were amplified by PCR. The PCR was performed using a common method under the following conditions. First, target genomic DNAs were diluted to a concentration of 2.5 ng/ml. Then the following PCR mixture was prepared.

| | |
|---|---|
| Water (HPLC grade) | 2.24 μl |
| 10x buffer (15 mM MgCl₂, 25 mM MgCl₂) | 0.5 μl |
| dNTP Mix (GIBCO) (25 mM for each) | 0.04 μl |
| Taq pol (HotStar) (5 U/μl) | 0.02 μl |
| Forward/reverse primer Mix (1 μM for each) | 0.02 μl |
| DNA | 1.00 μl |
| Total volume | 5.00 μl |

Here, the forward and reverse primers were designed based on upstream and downstream sequences of the SNPs from a known database. These primers are listed in Table 5 below.

The conditions of PCR were as follows: incubation at 95° C. for 15 minutes, at 95° C. for 30 seconds, at 56° C. for 30 seconds, and at 72° C. for 1 minute, repeated 45 times; and finally incubation at 72° C. for 3 minutes and storage at 4° C.

3. Analysis of SNPs in amplified target DNA fragments

Analysis of SNPs in the amplified target DNA fragments was performed using a homogeneous MassExtension (hME) technique available from Sequenom. The principle of the MassExtension technique is as follows. First, primers (also known as “extension primers”) ending immediately one base before SNPs within the target DNA fragments were designed. Then the primers were hybridized with the target DNA fragments and DNA polymerization was initiated. At this time, a polymerization solution contained a reagent (e.g., ddTTP) terminating the polymerization immediately after the incorporation of a nucleotide complementary to a first allelic nucleotide (e.g., A allele). In this regard, when the first allele (e.g., A allele) exists in the target DNA fragments, products in which only a nucleotide (e.g., T nucleotide) complementary to the first allele is extended from the primers are obtained. On the other hand, when a second allele (e.g., G allele) exists in the target DNA fragments, a nucleotide (e.g., C nucleotide) complementary to the second allele is added to the 3′-ends of the primers and then the primers are extended until a nucleotide complementary to the closest first allele nucleotide (e.g., A nucleotide) is added. The lengths of products extended from the primers were determined by mass spectrometry. In this way, alleles present in the target DNA fragments could be identified. Illustrative experimental conditions were as follows.

First, unreacted dNTPs were removed from the PCR products. For this, 1.53 μl of distilled water, 0.17 μl of HME buffer, and 0.30 μl of shrimp alkaline phosphatase (SAP) were added and mixed in 1.5 ml tubes to prepare SAP enzyme solutions. The tubes were centrifuged at 5,000 rpm for 10 seconds. Thereafter, the PCR products were added to the SAP solution tubes, sealed, incubated at 37° C. for 20 minutes and then at 85° C. for 5 minutes, and stored at 4° C.

Next, homogeneous extension was performed using the target DNA fragments as templates. The compositions of reaction solutions for the extension were as follows.

| | |
|---|---|
| Water (nanoscale distilled water) | 1.728 μl |
| hME extension mix (10x buffer containing 2.25 mM d/ddNTPs) | 0.200 μl |
| Extension primers (100 μM for each) | 0.054 μl |
| Thermosequenase (32 U/μl) | 0.018 μl |
| Total volume | 2.00 μl |

The reaction solutions were thoroughly stirred and subjected to spin-down centrifugation. Tubes or plates containing the resultant solutions were compactly sealed and incubated at 94° C. for 2 minutes, followed by 40 thermal cycles at 94° C. for 5 seconds, at 52° C. for 5 seconds, and at 72° C. for 5 seconds, and storage at 4° C. The homogeneous extension products thus obtained were washed with a resin (SpectroCLEAN™).

Nucleotides of polymorphic sites in the extension products were assayed using mass spectrometry, MALDI-TOF (Matrix Assisted Laser Desorption and Ionization-Time of Flight). The MALDI-TOF is operated according to the following principle. When an analyte is exposed to a laser beam, it flies toward a detector positioned at the opposite side in a vacuum state, together with an ionized matrix. At this time, the time taken for the analyte to reach the detector is calculated. A material with a smaller mass reaches the detector more rapidly. The nucleotides of SNPs in the target DNA fragments were determined based on a difference in mass between the DNA fragments and known SNP sequences. Primers used in the amplification and extension of the target DNAs are listed in Table 5 below.

TABLE 5

| | Amplification primer | | |
|---|---|---|---|
| | (SEQ ID NO.) | | Extension primer |
| SNP site | Forward primer | Reverse primer | (SEQ ID NO.) |

| | | | |
|---|---|---|---|
| CCM108 | 4 | 5 | 6 |
| CCM128 | 7 | 8 | 9 |

The results for the determination of polymorphic sequences of the target DNAs using the MALDI-TOF are shown in Table 2 above. Each allele may exist in the form of homozygote or heterozygote in an individual. However, in the population, the relative frequency of homozygote and heterozygote is statistically insignificant. According to Mendel's Law of inheritance and the Hardy-Weinberg Law, a genetic makeup of alleles constituting a population is maintained at a constant frequency. When the genetic makeup is statistically significant, it can be considered to be biologically meaningful.

A polynucleotide of the present invention can be used for colorectal cancer-related applications such as diagnosis, treatment, or fingerprinting analysis of colorectal cancer.

A microarray and diagnostic kit including the polynucleotide of the present invention can be effectively used for the detection of colorectal cancer.

A method of analyzing polymorphic sequences associated with colorectal cancer of the present invention can effectively detect the presence or a risk of colorectal cancer.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A polynucleotide comprising
at least 10 contiguous nucleotides of a nucleotide sequence selected from the group consisting of nucleotide sequences of SEQ ID NOS: 1 and 2, wherein the at least 10 contiguous nucleotides comprises a nucleotide at position 101 of the selected nucleotide sequence, or
a complement thereof.

2. A polynucleotide which hybridizes with the polynucleotide of claim 1 or the complement of the polynucleotide that hybridizes with the polynucleotide of claim 1.

3. The polynucleotide of claim 1, having a length of 10 to 100 nucleotides.

4. The polynucleotide of claim 1, being a primer or a probe.

5. A microarray comprising the polynucleotide of claim 1.

6. A diagnostic kit for the detection of colorectal cancer, which comprises the polynucleotide of claim 1.

7. A method of diagnosing colorectal cancer in an individual, which comprises:
determining a nucleotide present in the individual at a polymorphic site,
wherein the polymorphic site corresponds to position 101 within a polynucleotide of SEQ ID NO: 1 or 2, or the complement thereof.

8. The method of claim 7, wherein the determining the nucleotide of at the polymorphic site comprises:
obtaining a nucleic acid sample from the individual;
contacting the nucleic acid sample with a microarray on which the polynucleotide of claim 1 is immobilized such that specific hybridization; and
detecting a hybridization result.

9. The method of claim 7, wherein, when the determined nucleotide at the polymorphic site corresponding to position 101 of SEQ ID NO: 1 comprises a G or the determined nucleotide at the polymorphic site corresponding to position 101 of SEQ ID NO: 2 comprises a T, it is determined that the individual has a higher likelihood of being diagnosed as a colorectal cancer patient or as at risk of developing colorectal cancer.

10. The method of claim 7, further comprising
obtaining a nucleic acid sample from the individual.

11. A method of diagnosing colorectal cancer in an individual, which comprises:
determining the presence or absence in the individual of a recessive nucleotide at polymorphic site CCM108 or polymorphic site CCM128 in the human eEF1A1 gene,
wherein the presence of the recessive nucleotide at CCM108 or CCM128 identifies the individual as having a higher likelihood of being at risk of developing colorectal cancer.

12. The method of claim 11, wherein said determining comprises determining whether the individual is homozygous or heterozygous for the recessive nucleotide at polymorphic site CCM108 or polymorphic site CCM128 in the human eEF1A1 gene.
